(19) United States
(12) Patent Application Publication
(10) Pub. No.: US 2021/0260303 A1
(43) Pub. Date: Aug. 26, 2021

(54) MEDICATION DELIVERY DEVICE AND MEDICATION DELIVERY ASSEMBLY

(71) Applicant: Sanofi, Paris (FR)

(72) Inventors: Michael Schabbach, Frankfurt am Main (DE); Anna Baccaro, Frankfurt am Main (DE)

(21) Appl. No.: 16/973,815

(22) PCT Filed: Jun. 13, 2019
(86) PCT No.: PCT/EP2019/065550

§ 371 Date: Dec. 10, 2020

(30) Foreign Application Priority Data
Jun. 14, 2018 (EP) 18305727.2

(51) Int. Cl. A61M 5/315; A61M 5/24

(52) U.S. Cl. CPC A61M 5/31591; A61M 2005/1787; A61M 5/2466; A61M 5/31501

(57) ABSTRACT

A medication delivery device is disclosed. The medication delivery device comprises a housing, a drug container holder arranged at least partly within the housing, wherein the drug container holder is configured to receive a plurality of single dose drug containers each of which is configured to contain a liquid, and an actuator element configured to separately administer a single dose of the liquid from each of the plurality of single dose drug containers. Further, a medication delivery assembly, a method for manufacturing a medication delivery device and a method for administration a single dose of a liquid using a medication delivery device are disclosed.

### CROSS REFERENCE TO RELATED APPLICATIONS

The present application is the national stage entry of International Patent Application No. PCT/EP2019/065550, filed on Jun. 13, 2019, and claims priority to Application No. EP 18305727.2, filed on Jun. 14, 2018, the disclosures of which are incorporated herein by reference.

### TECHNICAL FIELD

The present disclosure relates to a medication delivery device and a medication delivery assembly. The present disclosure further relates to a method for manufacturing a medication delivery device and to a method for administration a single dose of a liquid using a medication delivery device.

### BACKGROUND

Many liquids such as medicaments have to be injected into the body. This applies in particular to medicaments, which are deactivated or have their efficiency remarkably decreased by oral administration, e.g. proteins (such as insulin, growth hormones, interferons), carbohydrates (e.g. heparin), antibodies and the majority of vaccines. Such medicaments are predominantly injected by means of delivery devices such as syringes, medicament pens or medicament pumps.

The user of such syringes, medicament pens or medicament pumps can range from healthcare professionals to the medicament-recipient themselves, the latter ranging from children or elderly persons. The medicinal injections may include repetitive or multiple injections of a particular dose (e.g. a vaccine in multi-dosage regimen) to a single injection of a single dose (e.g. a vaccine or in an emergency hydrocortisone).

For this purpose, there are several types of medication delivery devices known such as pen type delivery devices. These delivery devices have in common that they are configured to handle only one drug container containing the medical liquid to be delivered.

Despite the advantages provided by these delivery devices, there are still some drawbacks. Particularly, only in a few exceptions, it is necessary to deliver one dose of the medical liquid. In most cases, a user has to deliver a plurality of doses in a subsequent order such as seven doses in total with one dose per day or the like. The process of inserting each single container into the device and to discharge the same for replacement purposes is quite laborious.

### SUMMARY

Disclosed herein are a medication delivery device, a medication delivery assembly, a method for manufacturing a medication delivery device and a method for administration a single dose of a liquid using a medication delivery device allowing to facilitate the delivery process of a plurality of single doses or to make the same more convenient for an user.

Embodiments of the disclosed medication delivery device, medication delivery assembly, method for manufacturing a medication delivery device and method for administration a single dose of a liquid using a medication delivery device have the features of the independent claims. Particular embodiments, which might be realized in an isolated fashion or in any arbitrary combination, are listed in the dependent claims.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

The disclosed medication delivery device comprises a housing, a drug container holder arranged at least partly within the housing, wherein the drug container holder is configured to receive a plurality of single dose drug containers each of which is configured to contain a liquid, and an actuator element configured to separately administer a single dose of the liquid from each of the plurality of single dose drug containers.

The term “medication delivery device” as used herein refers to any device configured to deliver or administer a liquid from a respective container. Particularly, the medication delivery device is configured to deliver or administer a predetermined dose of a liquid.

The term “liquid” as used herein refers to any liquid which is provided in single dose drug container. The liquid may be a medical liquid or a dietary supplement such as a vitamin solution. The term “drug container holder” as used herein refers to any device configured to receive a plurality of single dose drug containers. The drug container holder may be a magazine in which the single dose drug containers are provided.

The term “actuator element” as used herein refers to any device or element configured to administer a single dose of the liquid from a single dose drug container. Thus, the actuator element may be any device or element configured to allow the liquid to be delivered or administered from the single dose drug container such as a piston.

With the disclosed medication delivery device, it is possible to deliver or administer a plurality of single doses of a liquid from a plurality of single dose drug containers in a subsequent order without the necessity to replace the container after use by a new one. Rather, only the container holder carrying the plurality of containers has to be inserted into the housing. Thus, with a single insertion process, a plurality of containers are supplied to the medication delivery device.

The drug container holder may comprise at least one guiding means so as to be configured to receive a plurality of rigid or flexible single dose drug containers and to guide the single dose drug containers to an administration position within the housing. Thus, the containers may be forwarded to a target position allowing to deliver the liquid therefrom.

For example, the guiding means is at least one rib and/or rail. Thus, with a rather simple constructional member, the containers may be forwarded to a target position allowing to deliver the liquid therefrom, which decreases the manufacturing costs.

The actuator element may be configured to separately administer the single dose through a hollow needle. Thus, the liquid may be administered by means of an injection or piercing process using the needle. Accordingly, the medication delivery device may be used with a broad range of applications.

The housing may be configured to allow the hollow needle to be removably connected to the housing. Thus, the housing is configured to use a single use or disposable needle.

The needle may comprise a connecting means configured to connect the needle to each of the plurality of single dose drug containers. The term “connecting means” as used herein refers to any device configured to cause a connection of the needle to the container. The connection may be realized by a positive fit or a negative fit connection. The connecting means may be a bulge, a clip, a snap-fit mechanism, a threaded connector, a bayonet fit mechanism, a protrusion-groove combination or the like. Thus, the container may be disposed together with the needle after use which reduces the risk of infection.

The hollow needle may be connected to each of the plurality of single dose drug containers. With other words, to each of the plurality of single dose drug containers a respective needle may be connected. Thus, the containers may be provided similar to a ready-to-use-kit.

The hollow needle may be at least partially arranged within each of the plurality of single dose drug containers. Thus, the risk of injuries caused by a protruding needle is significantly reduced.

The hollow needle may be configured to penetrate a pierceable portion of each of the plurality of single dose drug containers. Thus, the container may be opened by means of the needle which facilitates the opening process.

For example, the hollow needle may comprise a first end configured to penetrate a patient's skin and a second end configured to penetrate the pierceable portion. It is to be noted that the first and second ends are different ends of the needle. Thus, a fluid or liquid communication from the interior of the container and the outside thereof may be established.

The actuator element may be configured to move the hollow needle relative to each of the plurality of single dose drug containers. Thus, with a rather simple relative movement, the needle and the container, respectively, may be moved to a position allowing to establish a fluid or liquid communication from the interior of the container and the outside thereof.

Particularly, the actuator element may be configured to move the hollow needle relative to each of the plurality of single dose drug containers such that the hollow needle penetrates the pierceable portion. Thus, with a rather simple relative movement the needle may cause the container to be opened.

The pierceable portion may be a septum. Thus, the needle may penetrate a sterile member of the container which reduces the risk of any infection or contamination.

The needle may be substantially U-shaped. Thus, the needle may be designed rather compact with two ends thereof facing in the same direction. Alternatively, the needle may comprise a longitudinal shape. Thus, with both alternatives the container and a patient's skin may be pierced with a single movement. Accordingly, the total construction may be simplified.

The actuator element may be configured to move a moveable stopper of each of the plurality of single dose drug containers so as to cause the liquid of the single dose drug container to be discharged therefrom. Thus, the movement of the moveable stopper causes the liquid to flow out of the container.

A movement of the moveable stopper reduces an inner volume of the single dose drug container. Thus, the container may be squeezed so as to increase the amount of dischargeable liquid.

The drug container holder is configured to receive a magazine comprising the plurality of single dose drug containers. Thus, the plurality of containers may be provided in compact and well established device.

The magazine comprises a linear or circular arrangement of the plurality of single dose drug containers. Thus, the containers may be provided in an appropriate manner depending on the respective application.

The medication delivery device may further comprise a waste container configured to receive at least one used single dose drug container. Thus, the used container may be temporarily stored before being discarded. Thus, the user may select an appropriate point of time for removing the used container from the waste container.

The housing may be a reusable housing. Alternatively, the housing may be a disposable housing. Thus, the type of housing may be selected depending on the respective application.

The actuator element may be a piston or comprises a piston. Thus, the liquid may be administered by means of a well established constructional member which decreases the manufacturing costs.

The medication delivery device may further comprise an ejector configured to eject a used single dose drug container from the drug container holder. Thus, a used container may be automatically or semi-automatically ejected and has not to be manually ejected which avoids contact of the user with the used container.

The medication delivery device may further comprise a dose button configured to operate the actuator element. Thus, the actuation process is simplified for the user.

Each of the plurality of single dose containers may contain the liquid before being used. Thus, the containers are already filled and do not have to be prepared by the user before administration.

The disclosed medication delivery assembly comprises a medication delivery device as described before and a plurality of single dose drug containers. Thus, the assembly may be provided as a ready-to-use-kit.

The medication delivery assembly may further comprise a magazine, wherein the plurality of single dose drug containers is arranged in the magazine. Thus, the assembly may use a rather compact storage device for the containers which is convenient to the user.

The magazine comprises a linear or circular arrangement of the plurality of single dose drug containers. Thus, the containers may be provided in an appropriate manner depending on the respective application.

The medication delivery assembly may further comprise a needle. Thus, the liquid may be administered to a patient by means of an injection process which is a common or well known process. Accordingly, usually a patient in need thereof knows how to handle the assembly without any extensive studies of a user's manual.

The needle is configured to be removable connected to the housing. Thus, only the needle has to be replaced and not the total delivery device.

The needle may comprise a connecting means configured to connect the needle to each of the plurality of single dose drug containers. Thus, the container may be disposed together with the needle after use which reduces the risk of infection.

The needle may be connected to each of the plurality of single dose drug containers. Thus, the containers may be provided in a ready-to-use manner.

The needle may be substantially U-shaped. Thus, the needle may be designed rather compact with two ends thereof facing in the same direction. Thus, the container and a patient's skin may be pierced with a single movement. Accordingly, the total construction may be simplified.

Each of the plurality of single dose drug containers may further comprise a spring configured to bias the actuator element towards a start position in which administration of the single dose is prevented. Thus, any undesired or premature administration of the liquid is prevented.

The actuator element may be moveable by means of an actuation force in a direction opposite to a biasing direction of the spring. Thus, the liquid is administered by means of an actuation in a direction counter-acting the biasing spring.

The spring is configured to move a valve of each of the plurality of single dose drug containers to an open position, in which the medical liquid of the single dose drug container is allowed to be discharged therefrom. Thus, the valve is opened by means of an actuation in a direction counter-acting the biasing spring.

The disclosed method for manufacturing a medication delivery device, preferably a medication delivery device as described before, comprises: providing a housing, arranging a drug container holder at least partly within the housing, wherein the drug container holder is configured to receive a plurality of single dose drug containers each of which is configured to contain a liquid, and mounting an actuator element configured to separately administer a single dose of the liquid from each of the plurality of single dose drug containers.

Thus, the delivery device may be manufactured with only a few constructional members and with a few assembling steps.

The disclosed method for administration of a single dose of a liquid using a medication delivery device as described before comprises: inputting one single dose drug containers of a plurality of single dose drug containers into the drug container holder, wherein the one single dose drug container includes a liquid, and operating the actuator element so as to administer a single dose of the liquid from the one single dose drug container.

Thus, the administration process is simplified to the user.

The method for administration of a single dose of a liquid may further comprise discharging the one single dose drug container after administering the single dose. Thus, a used container is disposed after use without any manual step from the user's side.

The method for administration of a single dose of a liquid may further comprise inputting a subsequent one single dose drug containers of the plurality of single dose drug containers into the drug container holder. Thus, a used container is automatically or semi-automatically replaced by a new one.

The method for administration of a single dose of a liquid may further comprise administering the single dose of the liquid through a hollow needle. Thus, the liquid may be administered by means of an injection process which is a common or well known process. Accordingly, usually a patient in need thereof knows how to handle the assembly without any extensive studies of a user's manual.

Summarizing the findings of the present invention, the following embodiments are preferred:

Embodiment 1: A medication delivery device, comprising a housing, a drug container holder arranged at least partly within the housing, wherein the drug container holder is configured to receive a plurality of single dose drug containers each of which is configured to contain a liquid, and an actuator element configured to separately administer a single dose of the liquid from each of the plurality of single dose drug containers.

Embodiment 2: The medication delivery device according to embodiment 1, wherein the drug container holder comprises at least one guiding means so as to be configured to receive a plurality of rigid or flexible single dose drug containers and to guide the single dose drug containers to an administration position within the housing.

Embodiment 3: The medication delivery device according to embodiment 2, wherein the guiding means is at least one rib and/or rail.

Embodiment 4: The medication delivery device according to any one of embodiments 1 to 3, wherein the actuator element is configured to separately administer the single dose through a hollow needle.

Embodiment 5: The medication delivery device according to embodiment 4, wherein the housing is configured to allow the hollow needle to be removably connected to the housing.

Embodiment 6: The medication delivery device according to embodiment 4 or 5, wherein the needle comprises a connecting means configured to connect the needle to each of the plurality of single dose drug containers.

Embodiment 7: The medication delivery device according to embodiment 4, wherein the hollow needle is connected to each of the plurality of single dose drug containers.

Embodiment 8: The medication delivery device according to embodiment 7, wherein the hollow needle is at least partially arranged within each of the plurality of single dose drug containers.

Embodiment 9: The medication delivery device according to any one of embodiments 4 to 8, wherein the hollow needle is configured to penetrate a pierceable portion of each of the plurality of single dose drug containers.

Embodiment 10: The medication delivery device according to embodiment 9, wherein the hollow needle comprises a first end configured to penetrate a patient's skin and a second end configured to penetrate the pierceable portion.

Embodiment 11: The medication delivery device according to embodiment 9 or 10, wherein the actuator element is configured to move the hollow needle relative to each of the plurality of single dose drug containers.

Embodiment 12: The medication delivery device according to embodiment 11, wherein the actuator element is configured to move the hollow needle relative to each of the plurality of single dose drug containers such that the hollow needle penetrates the pierceable portion.

Embodiment 13: The medication delivery device according to any one of embodiments 10 to 12, wherein the pierceable portion is a septum.

Embodiment 14: The medication delivery device according to any one of embodiments 4 to 13, wherein the needle is substantially U-shaped or comprises a longitudinal shape.

Embodiment 15: The medication delivery device according to any one of embodiments 1 to 14, wherein the actuator element is configured to move a moveable stopper of each of the plurality of single dose drug containers so as to cause the liquid of the single dose drug container to be discharged therefrom.

Embodiment 16: The medication delivery device according to embodiment 15, wherein a movement of the moveable stopper reduces an inner volume of the single dose drug container.

Embodiment 17: The medication delivery device according to any one of embodiments 1 to 16, wherein the drug container holder is configured to receive a magazine comprising the plurality of single dose drug containers.

Embodiment 18: The medication delivery device according to embodiment 17, wherein the magazine comprises a linear or circular arrangement of the plurality of single dose drug containers.

Embodiment 19: The medication delivery device according to any one of embodiments 1 to 18, further comprising a waste container configured to receive at least one used single dose drug container.

Embodiment 20: The medication delivery device according to any one of embodiments 1 to 19, wherein the housing is a reusable or a disposable housing.

Embodiment 21: The medication delivery device according to any one of embodiments 1 to 20, wherein the actuator element is a piston or comprises a piston.

Embodiment 22: The medication delivery device according to any one of embodiments 1 to 21, further comprising an ejector configured to eject a used single dose drug container from the drug container holder.

Embodiment 23: The medication delivery device according to any one of embodiments 1 to 22, further comprising a dose button configured to operate the actuator element.

Embodiment 24: The medication delivery device according to any one of embodiments 1 to 23, wherein each of the plurality of single dose containers contains the liquid before being used.

Embodiment 25: A medication delivery assembly comprising a medication delivery device according to any one of embodiments 1 to 24 and a plurality of single dose drug containers.

Embodiment 26: The medication delivery assembly according to embodiment 25, further comprising a magazine, wherein the plurality of single dose drug containers are arranged in the magazine.

Embodiment 27: The medication delivery assembly according to embodiment 26, wherein the magazine comprises a linear or circular arrangement of the plurality of single dose drug containers.

Embodiment 28: The medication delivery assembly according to any one of embodiments 25 to 27, further comprising a needle.

Embodiment 29: The medication delivery assembly according to embodiment 28, wherein the needle is configured to be removable connected to the housing.

Embodiment 30: The medication delivery assembly according to embodiment 28 or 29, wherein the needle comprises a connecting means configured to connect the needle to each of the plurality of single dose drug containers.

Embodiment 31: The medication delivery assembly according to embodiment 28, wherein the needle is connected to each of the plurality of single dose drug containers.

Embodiment 32: The medication delivery assembly according to embodiment 31, wherein the needle is substantially U-shaped.

Embodiment 33: The medication delivery assembly according to any one of embodiments 25 to 32, wherein each of the plurality of single dose drug containers further comprises a spring configured to bias the actuator element towards a start position in which administration of the single dose is prevented.

Embodiment 34: The medication delivery assembly according to embodiment 33, wherein the actuator element is moveable by means of an actuation force in a direction opposite to a biasing direction of the spring.

Embodiment 35: The medication delivery assembly according to embodiment 33 or 34, wherein the spring is configured to move a valve of each of the plurality of single dose drug containers to an open position, in which the medical liquid of the single dose drug container is allowed to be discharged therefrom.

Embodiment 36: Method for manufacturing a medication delivery device, preferably a medication delivery device according to any one of embodiments 1 to 24, comprising: providing a housing, arranging a drug container holder at least partly within the housing, wherein the drug container holder is configured to receive a plurality of single dose drug containers each of which is configured to contain a liquid, and mounting an actuator element configured to separately administer a single dose of the liquid from each of the plurality of single dose drug containers.

Embodiment 37: Method for administration a single dose of a liquid using a medication delivery device according to any one of embodiments 1 to 24, comprising: inputting one single dose drug containers of a plurality of single dose drug containers into the drug container holder, wherein the one single dose drug container includes a liquid, and operating the actuator element so as to administer a single dose of the liquid from the one single dose drug container.

Embodiment 38: Method according to embodiment 37, further comprising discharging the one single dose drug container after administering the single dose.

Embodiment 39: Method according to embodiment 38, further comprising inputting a subsequent one single dose drug containers of the plurality of single dose drug containers into the drug container holder.

Embodiment 40: Method according to any one of embodiments 37 to 38, further comprising administering the single dose of the liquid through a hollow needle.

Further features and embodiments of the invention will be disclosed in more detail in the subsequent description of embodiments, particularly in conjunction with the dependent claims. Therein, the respective features may be realized in an isolated fashion as well as in any arbitrary feasible combination, as the skilled person will realize. The scope of the invention is not restricted by the embodiments. The embodiments are schematically depicted in the figures.

Therein, identical reference numbers in these figures refer to identical or functionally comparable elements.

### BRIEF DESCRIPTION OF THE FIGURES

FIGS. 1A to 1H show cross-sectional views of a medication delivery assembly according to a first embodiment in different operation states;

FIG. 2A shows a perspective view of a medication delivery assembly according to a second embodiment;

FIGS. 2B to 2I show cross-sectional views of the medication delivery assembly according to the second embodiment in different operation states;

FIGS. 3A to 3D show cross-sectional views of a container used with the medication delivery assembly according to the second embodiment in different operation states;

FIGS. 4A to 4G show side views of a medication delivery assembly according to a third embodiment in different operation states;

FIG. 5 shows a perspective view of a container used with the medication delivery assembly according to the third embodiment;

FIGS. 6A to 6C show cross-sectional views of a medication delivery assembly according to a fourth embodiment in different operation states; and

FIGS. 7A to 7E show cross-sectional views of a medication delivery assembly according to a fifth embodiment in different operation states.

### DETAILED DESCRIPTION

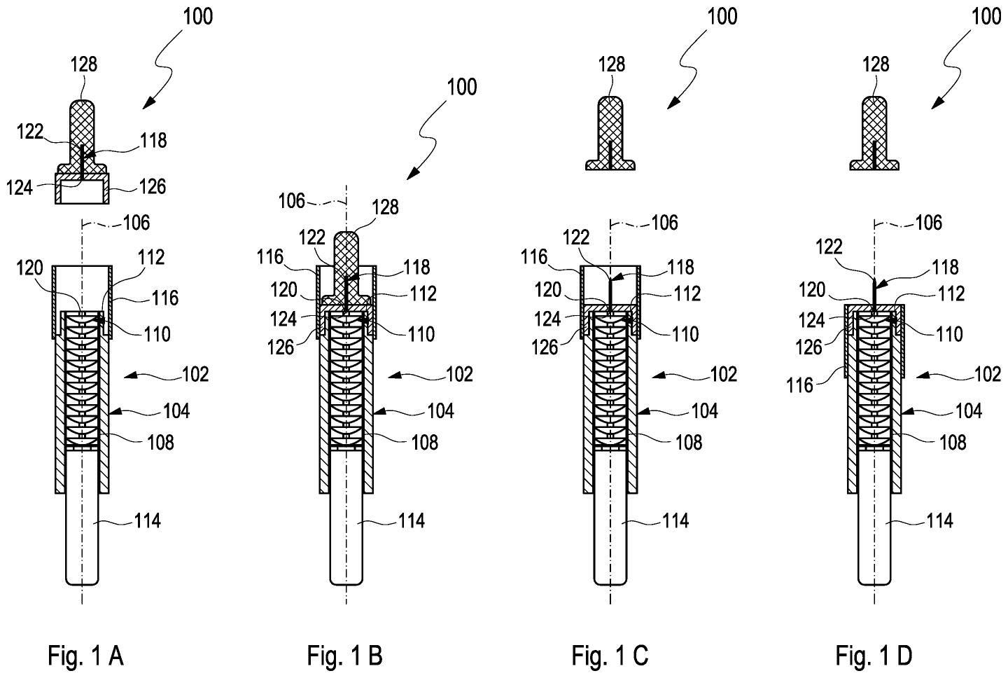

FIGS. 1A to 1H show cross-sectional views of a medication delivery assembly 100 according to a first embodiment in different operation states. It is to be noted that the operation starts with the state shown in FIG. 1A and terminates with the state shown in FIG. 1H. The medication delivery assembly 100 comprises a medication delivery device 102. The medication delivery device 102 comprises a housing 104. The housing 104 is substantially cylindrically formed. Thus, the housing 104 extends along a longitudinal axis 106, which is also an axis of symmetry of the housing 104. The housing 104 is a reusable housing 104.

The medication delivery device 102 further comprises a drug container holder 108. The drug container holder 108 is substantially cylindrically formed. The drug container holder 108 is at least partly arranged within the housing 104. In this embodiment, the drug container holder 108 is formed as a cylindrical sleeve inserted into the housing 104. The drug container holder 108 is configured to receive a plurality of single dose drug containers 110 each of which is configured to contain a liquid. Needless to say, the single dose drug containers 110 contain the liquid before being used. The liquid may be a medical liquid. The single dose drug containers 110 are part of the medication delivery assembly 100. The single dose drug containers 110 are flexible single dose drug containers 110. In this embodiment, the single dose drug containers 110 are formed similar to pouches and are arranged with one on the top of the other within the drug container holder 108. Particularly, the single dose drug containers 110 are arranged that the lower side of one single dose drug container 110 contacts the upper side of the adjacent one single dose drug container 110. The drug container holder 108 comprises at least one guiding means (not shown in detail) so as to be configured to receive a plurality of rigid or flexible single dose drug containers 110 and to guide the single dose drug containers 110 to an administration position within the housing 104. The guiding means is at least one rib and/or rail. For example, the guiding means may be inclined ribs configured to guide the single dose drug containers 110 towards a front end 112 of the housing 104, wherein the guiding means also prevents a movement of the single dose drug containers 110 in an opposite or rearwards direction.

The medication delivery device 102 further comprises an actuator element 114 configured to separately administer a single dose of the liquid from each of the plurality of single dose drug containers 110. The actuator element 114 is a piston. The medication delivery device 102 may further comprises a dose button (not shown in detail) configured to operate the actuator element 114. The medication delivery device 102 further comprises a moveable needle shield 116 located at the front end 112 of the housing 104. The actuator element 114 is configured to separately administer the single dose through a hollow needle 118. The needle 118 comprises a longitudinal shape. The housing 104 is configured to allow the hollow needle 118 to be removably connected to the housing 104. For example, the housing 104 comprises a clip (not shown in detail) engaging the needle 118. The actuator element 114 is configured to move the hollow needle 118 relative to each of the plurality of single dose drug containers 110. The hollow needle 118 is configured to penetrate a pierceable portion 120 of each of the plurality of single dose drug containers 110. More particularly, the actuator element 114 is configured to move the hollow needle 118 relative to each of the plurality of single dose drug containers 110 such that the hollow needle 118 penetrates the pierceable portion 120. Particularly, the hollow needle 118 comprises a first end 122 configured to penetrate a patient's skin and a second end 124 configured to penetrate the pierceable portion 120. The needle 118 further comprises a connecting means 126 configured to connect the needle 118 to each of the plurality of single dose drug containers 110. For example, the needle 118 comprises a bulge (not shown in detail) engaging a depression of the container 110. Before being used, the needle 118 is provided with a needle cap 128 in order to avoid an injury of a user.

Next, an operation of the medication delivery assembly 100 according to the first embodiment is described. The operation may be used in association with a method of administration a single dose of a liquid using the medication delivery device 102.

As shown in FIG. 1A, the drug container holder 108 holding a plurality of single dose containers 110 is inserted into the housing 104. Further, the needle cap 128 is attached to the needle 118. The needle 118 is separate from the housing 104. Subsequently, as shown in FIG. 1B, the needle 118 is connected to the front end 112 of the housing. For example, the needle 118 is clipped to the housing 104. The needle cap 128 is still arranged on the needle 118. Subsequently, as shown in FIG. 1C, the needle cap 128 is removed or detached from the needle 118. Subsequently, as shown in FIG. 1D, the medication delivery device 102 is disposed with the front end 112 of the housing 104 on a patient's skin. More particularly, the needle shield 116 is pushed to the patient's skin such that the needle shield 116 is moved rearwards and retracts from the housing 104. Thereby, the needle 118 protrudes from the housing 104 and pierces the patient's skin with the first end 122. Subsequently, as shown in FIG. 1E, the user operates the actuator element 114, wherein the actuator element 114 and the front most single dose container 110 are moved towards the front end 112 and the needle 118 pierces the to pierceable portion 120 of the front most single dose container 110 with the second end 124. Thus, the liquid is discharged from the front most single dose container 110 and a single dose of the liquid is administered to the patient through the hollow needle 118 until the front most single dose container 110 is empty. Subsequently, as shown in FIG. 1F, the medication delivery device 102 is removed from the patient's skin. Thereby, the needle shield 116 is moved forwards again so as to protrude from the front end 112 of the housing 104. Thus, the needle 118 still connected to the housing 104 is not exposed but shielded by the needle shield 116. Subsequently, as shown in FIG. 1G, the needle cap 128 is attached to the needle 118 again. Further, the needle 118 is connected to the front most single dose container 110 by means of the connecting means 126. Finally, as shown in FIG. 1H, the used needle 118 is removed from the housing 104 together with the used front most single dose container 110 by removing the needle cap 128. Thus, the front most single dose drug container 110 is discharged after administering the single dose. Thereafter, a subsequent one single dose drug container 110 of the plurality of single dose drug containers 110 is input into the drug container holder 108 at a position allowing administration of the liquid thereof. Thereby, the operation described before may be repeated until all single dose drug containers 110 provided in the container holder 108 have been used. The medication delivery device 102 may further comprise an ejector (not shown in detail) configured to eject a used single dose drug container 110 from the drug container holder 108.

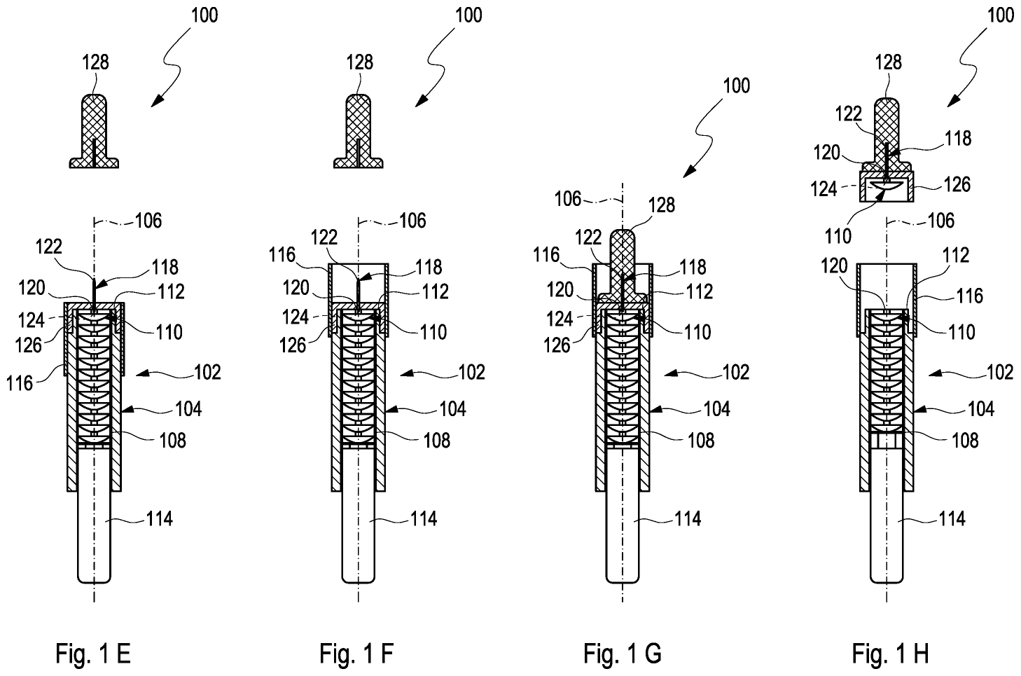

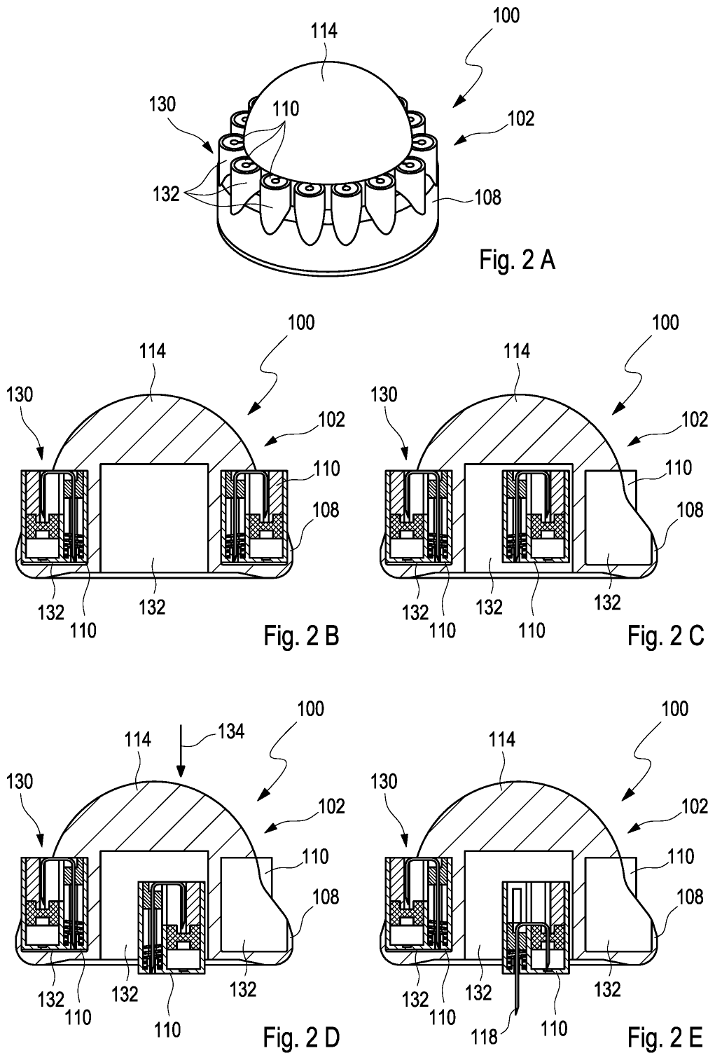

FIG. 2A shows a perspective view of a medication delivery assembly 100 according to a second embodiment. FIGS. 2B to 2I show cross-sectional views of the medication delivery assembly 100 according to the second embodiment in different operation states. Hereinafter, only the differences from the first embodiment will be described and like constructional members are indicated by like reference numerals. It is to be noted that the housing 104 of the medication delivery device 102 is not illustrated for illustration reasons. The container holder 108 is configured to receive a magazine 130 comprising the plurality of single dose drug containers 110. The magazine 130 comprises a circular arrangement of the plurality of single dose drug containers 110. For this purpose, the magazine 130 comprises a plurality of compartments 132 which are arranged on an imaginary circle around a central compartment 132 at a middle point of the imaginary circle. The actuator element 114 is a dome.

Next, an operation of the medication delivery assembly 100 according to the second embodiment is described. The operation may be used in association with a method of administration a single dose of a liquid using the medication delivery device 102.

As shown in FIG. 2B, the container holder 108 is provided with the magazine 130 holding a plurality of single dose drug containers 110. All compartments 132 of the magazine 130 except for the central compartment 132 are provided with the plurality of single dose drug containers 110. Subsequently, as shown in FIG. 2C, the magazine 130 is rotated to a predetermined position. Then, one of the single dose drug containers 110 is moved to the central compartment 132. The one of the single dose drug containers 110 may be moved to the central compartment 132 by means of a plunger (not shown in detail) or the like. Subsequently, as shown in FIG. 2D, the actuator element 114 is operated in the direction indicated by arrow 134 and the guiding means of the drug container holder 108 guides the one single dose drug container 110 to an administration position within the housing 104. Thereby, the needle 118 is moved relative to the one single dose drug container 110 as shown in FIG. 2E and the needle 118 protrudes from the housing 104. Subsequently, as shown in FIG. 2F, the needle 118 pierces the pierceable portion 120 and the liquid is discharged from the one single dose drug container 110 through the needle 118 until the one single dose drug container 110 is empty. Subsequently, as shown in FIG. 2G, the actuation force on the actuator element 114 is released and the needle 118 retracts into the one single dose drug container 110. Subsequently, as shown in FIG. 2H, the one single dose drug container 110 is moved back into the central compartment 132 of the magazine 130. Finally, as shown in FIG. 2I, the one single dose drug container 110 is discharged from the central compartment 132 of the magazine 130. Thus, the one single dose drug container 110 is discharged after administering the single dose. Thereafter, a subsequent one single dose drug container 110 of the plurality of single dose drug containers 110 is input into the drug container holder 108 at a position allowing administration of the liquid thereof. Thereby, the operation described before may be repeated until all single dose drug containers 110 provided in the container holder 108 and the magazine 130, respectively, have been used.

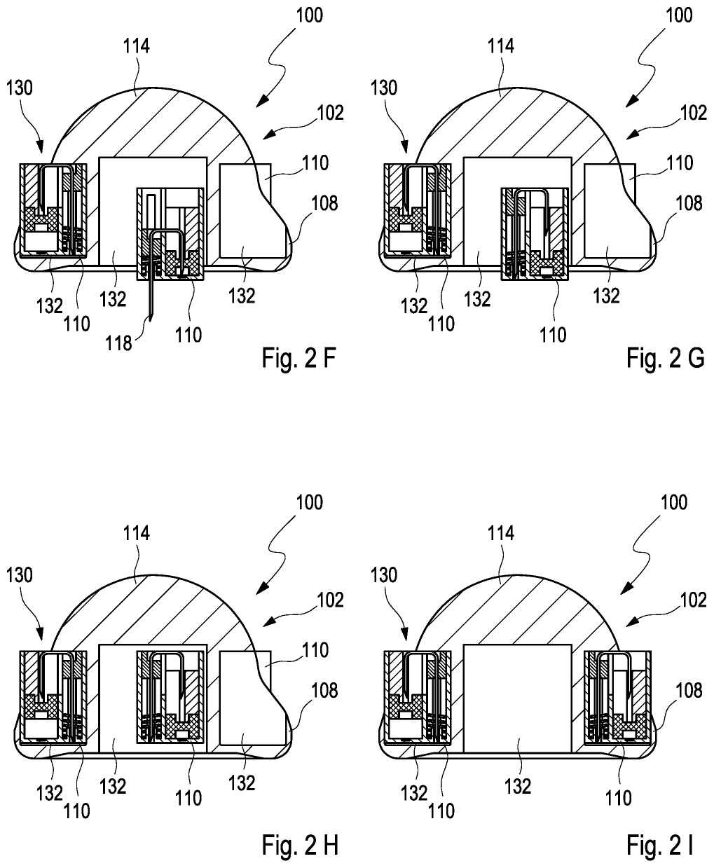

FIGS. 3A to 3D show cross-sectional views of a single dose drug container 110 used with the medication delivery assembly 100 according to the second embodiment in different operation states. Thus, further details of the operation of the single dose drug container 110 during the operation shown in FIGS. 2B to 2I will become more apparent. The single dose drug container 110 comprises a front end 136 and a rear end 138. The pierceable portion 120 is located at the front end 136. The pierceable portion 120 is a septum. The single dose drug container 110 further comprises a spring 140, a needle mount 142, a moveable stopper 144 and a stopper mount 146. The moveable stopper 144 is mounted to the stopper mount 146. The needle mount 142 and the stopper mount 146 are arranged adjacent to one another such as parallel. The needle mount 142 and the moveable stopper 144 together with the stopper mount 146 are respectively linearly moveable between a first position adjacent the rear end 138 and a second position adjacent the front end 136. The spring 140 is configured to bias the needle mount 142 towards the first position. The spring 140 is further configured to bias the actuator element 114 towards a start position in which administration of the single dose is prevented. Thus, the actuator element 114 is moveable by means of an actuation force in a direction opposite to a biasing direction of the spring 140. Further, the needle 118 is substantially U-shaped. The needle 118 is mounted to the needle mount 142 such that the first end 122 extends through the spring 140 and the second end 124 is located within a slit 148 extending through the stopper mount 146 and the moveable stopper 144. Thus, the needle 118 is connected to the single dose drug container 110. Particularly, the needle 118 is permanently connected to the single dose drug container 110. Further, the needle 118 is at least partially arranged within each of the plurality of single dose drug containers 110. A movement of the needle mount 142 causes a movement of the needle 118. The stopper mount 146 and the moveable stopper 144 are separately or independent moveable from the needle mount 142. The actuator element 114 is configured to act on the needle mount 142 so as to move the same. The actuator element 114 is also configured to act on the stopper mount 146 so as to move the same. Particularly, the actuator element 114 is configured to move the moveable stopper 144 of each of the plurality of single dose drug containers 110 so as to cause the liquid of the single dose drug container 110 to be discharged therefrom. A movement of the moveable stopper 144 reduces an inner volume of the single dose drug container 110 as will be explained in further detail below. The liquid is stored between walls of the singled dose drug container 110 and the moveable stopper 144.

As shown in FIG. 3A, before operating the actuator element 114, the needle mount 142 and the stopper mount 146 are respectively in the first position and the needle 118 does not protrude from the single dose drug container 110 due to the biasing force of the spring 140. Actuation of the actuator element 114 against the biasing force of the spring 140 first causes the needle mount 142 to move towards the second position as shown in FIG. 3B while the stopper mount 146 temporarily remains in the first position. Thereby, the needle 118 pierces the pierceable portion 120 with the first end 122 and protrudes from the single dose drug container 110. Further, the needle 118 enters the liquid within the single dose drug container 110 with the second end. Thereby, a fluid communication between the first end 122 and the second end 124 of the needle 118 is established. Subsequently, as shown in FIG. 3C, the actuator element 114 acts on the stopper mount 146 such that the stopper mount 146 and the moveable stopper 144 move towards the second position. The movement of the moveable stopper 144 reduces the inner volume of the single dose drug container 110 such that the liquid is forced to flow through the needle 118 from the second end 124 thereof to the first end 122 thereof so as to be discharged from the single dose drug container 110. Finally, as shown in FIG. 3D, the actuation force of the actuator element 114 is released. Thereby, the spring 140 biases the needle mount 142 towards the first position such that the needle 118 is retracted into the single dose drug container 110. Thus, after administration of the liquid, the needle 118 does not protrude anymore from the single dose drug container 110. The stopper mount 146 and the moveable stopper 144 may remain in the second position.

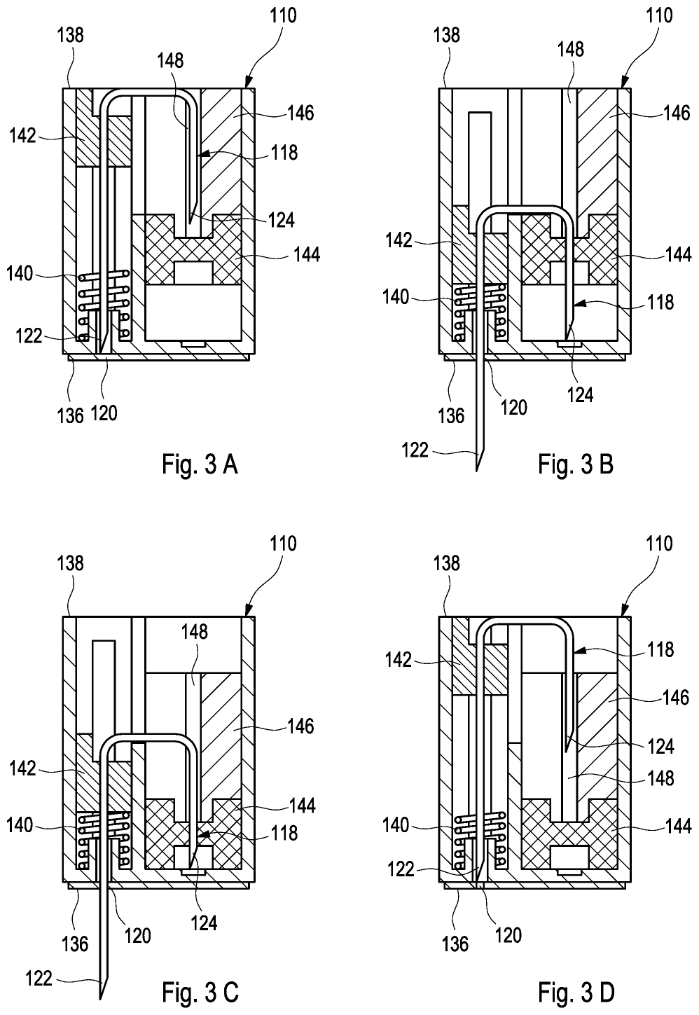

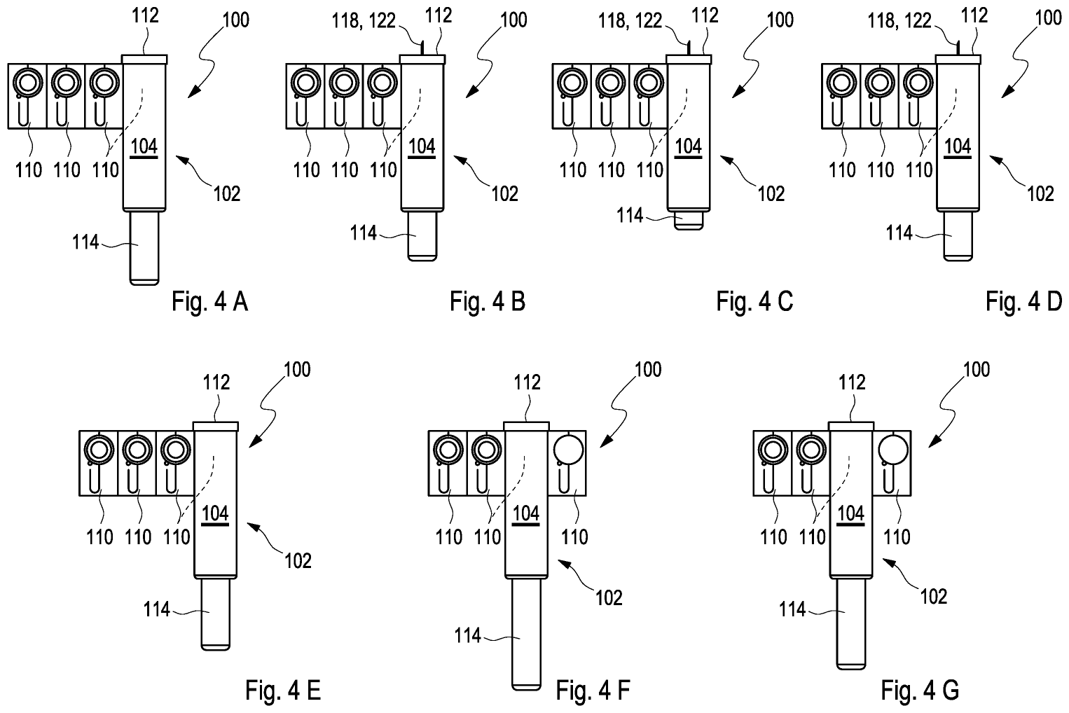

FIGS. 4A to 4G show side views of a medication delivery assembly 100 according to a third embodiment in different operation states. Hereinafter, only the differences from the previous embodiments will be described and like constructional members are indicated by like reference numerals. The single dose drug containers 110 are flexible single dose drug containers 110 which are arranged in a row with lateral portions adjacent to one another. Thus, the single dose drug containers 110 are similar to pouches. The single dose drug containers 110 are supplied into the container holder 108 in a lateral direction, i.e. perpendicular to the longitudinal axis 106 of the housing 104. Further, the needle 118 is substantially U-shaped. Particularly, the hollow needle 118 comprises a first end 122 configured to penetrate a patient's skin and a second end 124 (shown in FIG. 5) configured to penetrate the pierceable portion 120 (shown in FIG. 5).

Next, an operation of the medication delivery assembly 100 according to the third embodiment is described. The operation may be used in association with a method of administration a single dose of a liquid using the medication delivery device 102.

As shown in FIG. 4A, the container holder 108 is provided with the plurality of single dose drug containers 110. Particularly, one of the single dose drug containers 110 is input in the container holder 108. Subsequently, as shown in FIG. 4B, the actuator element 114 is operated and the guiding means of the drug container holder 108 guides the one single dose drug container 110 to an administration position within the housing 104. Thereby, the needle 118 is moved relative to the one single dose drug container 110 and the needle 118 protrudes from the housing 104 with the first end 122. Subsequently, referring to FIG. 4C, the needle 118 (shown in FIG. 5) pierces the pierceable portion 120 (shown in FIG. 5) with the second end 124 and the liquid is discharged from the one single dose drug container 110 through the needle 118 until, referring to FIG. 4D, the one single dose drug container 110 is empty. Subsequently, as shown in FIG. 4E, the actuation force on the actuator element 114 is released and the needle 118 retracts into the one single dose drug container 110. Subsequently, as shown in FIG. 4F, the one single dose drug container 110 is disposed from the container holder 108 and the next single dose drug container 110 is input into the container holder 108 at a position allowing administration of the liquid thereof. Thus, the one single dose drug container 110 is discharged after administering the single dose. Finally, as shown in FIG. 4G, the operation described before may be repeated until all single dose drug containers 110 provided in the container holder 108 have been used.

FIG. 5 shows a perspective view of a single dose drug container 110 used with the medication delivery assembly 100 according to the third embodiment. Thus, further details of the operation of the single dose drug container 110 during the operation shown in FIGS. 4A to 4G will become more apparent. The single dose drug container comprises a reservoir 150 containing the liquid. The single dose drug container 110 further comprises a front end 136 and a rear end 138. The pierceable portion 120 is located at the front end 136. The reservoir 150 is located adjacent the front end 136. The needle 118 is at least partially arranged within each of the plurality of single dose drug containers 110. Particularly, the needle 118 is permanently connected to the single dose drug container 110. The needle 118 partially extends below the reservoir 150. The actuator element 114 is configured to act on the needle 118 so as to move the same in a direction indicated by arrow 152. A movement of the needle 118 in the direction indicated by the arrow 152 causes the needle 118 to pierce the pierceable portion 120 with the first end 122 while the second end 124 enters the reservoir 150 at a position 154 facing away from the pierceable portion 120. Thereby, a fluid communication between the first end 122 and the second end 124 of the needle 118 is established and the liquid may be discharged from the single dose drug container 110.

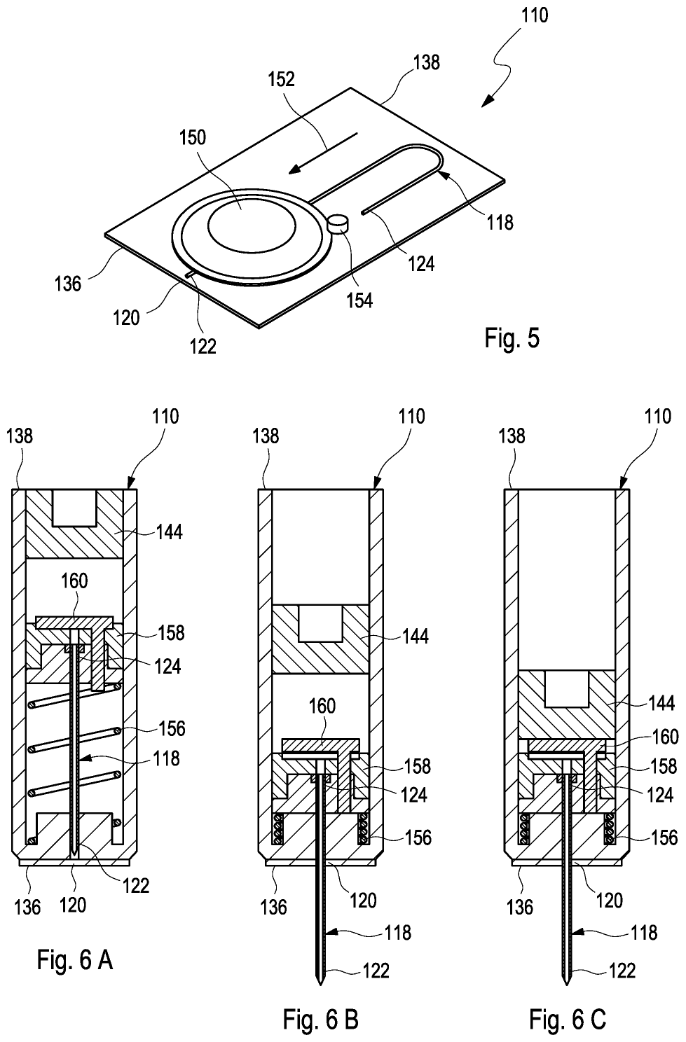

FIGS. 6A to 6C show cross-sectional views of a medication delivery assembly 100 according to a fourth embodiment in different operation states. Hereinafter, only the differences from the previous embodiments will be described and like constructional members are indicated by like reference numerals. The single dose drug container 110 comprises a front end 136 and a rear end 138. The pierceable portion 120 is located at the front end 136. The single dose drug container 110 further comprises a spring 156, a fixed stopper 158, a moveable stopper 144 and a valve 160. The needle 118 comprises a longitudinal shape and extends through the spring 156 with the first end 122. The needle 118 is mounted to the fixed stopper 158. The liquid is stored between the fixed stopper 158 and the moveable stopper 144. The valve 160 is arranged at the fixed stopper 158. The fixed stopper 158 is located closer to the front end 136 than the moveable stopper 144. The fixed stopper 158 and the moveable stopper 144 are respectively linearly moveable between a first position adjacent the rear end 138 and a second position adjacent the front end 136. The moveable stopper 144 is moveable relative to the fixed stopper 158.

The spring 156 is configured to bias the fixed stopper 158 towards the first position. Further, the needle 118 comprises a longitudinal shape. The needle 118 is mounted to the fixed stopper 158 such that the first end 122 extends through the spring 156 and the second end 124 is located at the valve 160. Thus, the needle 118 is connected to the single dose drug container 110. Particularly, the needle 118 is permanently connected to the single dose drug container 110. Further, the needle 118 is at least partially arranged within each of the plurality of single dose drug containers 110. A movement of the fixed stopper 158 causes a movement of the needle 118. The actuator element 114 is configured to act on the moveable stopper 144 so as to move the same. Particularly, the actuator element 114 is configured to move the moveable stopper 144 of each of the plurality of single dose drug containers 110 so as to cause the liquid of the single dose drug container 110 to be discharged therefrom. A movement of the moveable stopper 144 reduces an inner volume of the single dose drug container 110 as will be explained in further detail below. The liquid is stored between walls of the singled dose drug container 110, the fixed stopper 158 and the moveable stopper 144. A movement of the moveable stopper 144 towards the fixed stopper 158 causes the valve 160 to open such that the liquid may be discharged or administered through the hollow needle 118. Thus, the spring 156 is configured to bias the actuator element 114 towards a start position in which administration of the single dose is prevented. Further, the actuator element 114 is moveable by means of an actuation force in a direction opposite to a biasing direction of the spring 156. The spring 156 is configured to move the valve 160 of each of the plurality of single dose drug containers 110 to an open position, in which the liquid of the single dose drug container 110 is allowed to be discharged therefrom.

Next, an operation of the medication delivery assembly 100 according to the fourth embodiment is described. The operation may be used in association with a method of administration a single dose of a liquid using the medication delivery device 102.

As shown in FIG. 6A, the container holder 108 is provided with the plurality of single dose drug containers 110. Particularly, one of the single dose drug containers 110 is input in the container holder 108. Before operating the actuator element 114, the moveable stopper 144 and the fixed stopper 158 are in the first position. Particularly, the moveable stopper 144 is adjacent the rear end 138. Further, the needle 118 does not protrude from the single dose drug container 110. Actuation of the actuator element 114 first causes the moveable stopper 144 to move towards the second position as shown in FIG. 6B. The pressure acting on the liquid via the moveable stopper 144 causes to fixed stopper 158 also to move to the second position. Thereby, the needle 118 pierces the pierceable portion 120 with the first end 122 and protrudes from the single dose drug container 110. Further, valve 160 opens due to the biasing force of the spring 156 such that the liquid enters the needle 118 at the second end. Particularly, a small gap (not shown in detail) between the valve 160 and the fixed stopper 158 is generated. Thereby, a fluid communication between the first end 122 and the second end 124 of the needle 118 is established. Furthermore, the fixed stopper 158 compresses the spring 156. Subsequently, as shown in FIG. 6C, the actuator element 114 further moves the moveable stopper 144 towards the fixed stopper 158 which in turn may not be moved further. Thus, the movement of the moveable stopper 144 reduces the inner volume of the single dose drug container 110 such that the liquid is forced to flow through the needle 118 from the second end 124 thereof to the first end 122 thereof so as to be discharged from the single dose drug container 110. Finally, as is not shown in further detail, the actuation force of the actuator element 114 is released. Thereby, the spring 156 biases the fixed stopper 158 together with the moveable stopper 144 towards the first position such that the needle 118 is retracted into the single dose drug container 110. Thus, after administration of the liquid, the needle 118 does not protrude anymore from the single dose drug container 110.

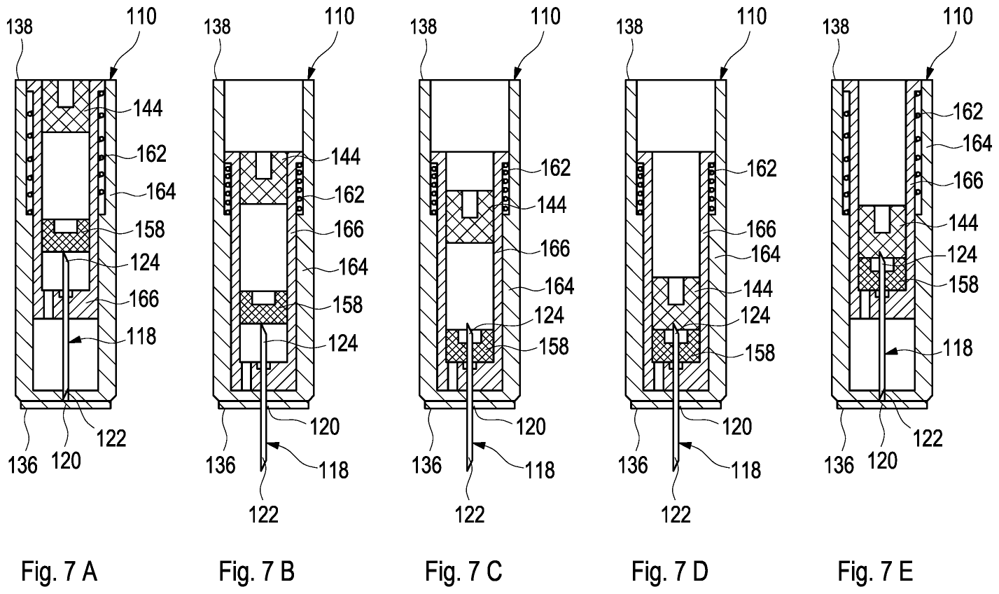

FIGS. 7A to 7E show cross-sectional views of a medication delivery assembly according to a fifth embodiment in different operation states. Hereinafter, only the differences from the previous embodiments will be described and like constructional members are indicated by like reference numerals. The single dose drug container 110 comprises a front end 136 and a rear end 138. The pierceable portion 120 is located at the front end 136. The single dose drug container 110 further comprises a spring 162. The spring 162 is configured to bias the actuator element 114 towards a start position in which administration of the single dose is prevented. The actuator element 114 is moveable by means of an actuation force in a direction opposite to a biasing direction of the spring 162. The single dose drug container 110 further comprises an outer casing 164 and an inner casing 166 having a fixed stopper 158 and a moveable stopper 144. The inner casing 166 is arranged within the outer casing 164 and is moveable relative to the outer casing 164. The spring 162 is arranged between the outer casing 164 and the inner casing 166 and abuts both of them. The needle 118 comprises a longitudinal shape and extends through the spring 156 with the first end 122. The needle 118 is mounted to the fixed stopper 158 at a side thereof facing the front end 136. The liquid is stored between the fixed stopper 158 and the moveable stopper 144. The fixed stopper 158 is located closer to the front end 136 than the moveable stopper 144. The fixed stopper 158 and the moveable stopper 144 are respectively linearly moveable between a first position adjacent the rear end 138 and a second position adjacent the front end 136. The moveable stopper 144 is moveable relative to the fixed stopper 158.

The spring 162 is configured to bias the inner casing 166 towards the rear end 138. Further, the needle 118 comprises a longitudinal shape. The needle 118 is mounted to the fixed stopper 158 such that the first end 122 faces the pierceable portion 120 and the second end 124 is located at the fixed stopper 158. Thus, the needle 118 is connected to the single dose drug container 110. Particularly, the needle 118 is permanently connected to the single dose drug container 110. Further, the needle 118 is at least partially arranged within each of the plurality of single dose drug containers 110. A movement of the fixed stopper 158 causes a movement of the needle 118. The actuator element 114 is configured to act on the moveable stopper 144 so as to move the inner casing 166 and subsequently the moveable stopper 144. Particularly, the actuator element 114 is configured to move the moveable stopper 144 of each of the plurality of single dose drug containers 110 so as to cause the liquid of the single dose drug container 110 to be discharged therefrom. A movement of the moveable stopper 144 reduces an inner volume of the single dose drug container 110 as will be explained in further detail below. The liquid is stored between walls of the inner casing 166 of the singled dose drug container 110, the fixed stopper 158 and the moveable stopper 144. A movement of the moveable stopper 144 towards the fixed stopper 158 causes the needle 118 to pierce the pierceable portion 120 with the first end 122 and the fixed stopper 158 with the second end 124 such that the liquid may be discharged or administered through the hollow needle 118.

Next, an operation of the medication delivery assembly 100 according to the fifth embodiment is described. The operation may be used in association with a method of administration a single dose of a liquid using the medication delivery device 102.

As shown in FIG. 7A, the container holder 108 is provided with the plurality of single dose drug containers 110. Particularly, one of the single dose drug containers 110 is input in the container holder 108. Before the actuator element 114 is operated, the inner casing 166 is at the rear end 138. Further, the moveable stopper 144 and the fixed stopper 158 are in the first position. Particularly, the moveable stopper 144 is adjacent the rear end 138. Further, the needle 118 does not protrude from the single dose drug container 110. Actuation of the actuator element 114 first causes the inner casing 166 to move within the outer casing 164 towards the front end 136. The actuator element 114 acts against the biasing force of the spring 162, which is compressed. The pressure acting on the liquid via the moveable stopper 144 causes to fixed stopper 158 to move to the second position. Thereby, the needle 118 pierces the pierceable portion 120 with the first end 122 and protrudes from the single dose drug container 110 as shown in FIG. 7B. Further actuation of the actuator element 114 causes the moveable stopper 144 to move towards the second position. Thuy, the needle 118 pierces and penetrates the fixed stopper 158 with the second end 124 as shown in Figured 7C. Thereby, a fluid communication between the first end 122 and the second end 124 of the needle 118 is established. Subsequently, as shown in FIG. 7D, the actuator element 114 further moves the moveable stopper 144 towards the fixed stopper 158 which in turn may not be moved further. Thus, the movement of the moveable stopper 144 reduces the inner volume of the single dose drug container 110 such that the liquid is forced to flow through the needle 118 from the second end 124 thereof to the first end 122 thereof so as to be discharged from the single dose drug container 110. Finally, as shown in FIG. 7E, the actuation force of the actuator element 114 is released. Thereby, the spring 162 biases the inner casing 166 within the outer casing 164 to the rear end 138. The fixed stopper 158 moves together with the needle 118 sticking thereto such that the needle 118 is retracted into the single dose drug container 110. Thus, after administration of the liquid, the needle 118 does not protrude anymore from the single dose drug container 110.

All embodiments for the medication delivery device 102 described before may be manufactured as will be described in further detail below. First, the housing 104 is provided. Then, the drug container holder 108 is arranged at least partly within the housing 104. The drug container holder 108 is configured to receive a plurality of single dose drug containers 110 each of which is configured to contain a liquid. Subsequently, the actuator element 114 configured to separately administer a single dose of the liquid from each of the plurality of single dose drug containers 110 is mounted to the housing 104.

### LIST OF REFERENCE NUMBERS

100 medication delivery assembly
102 medication delivery device
104 housing
106 longitudinal axis
108 drug container holder
110 single dose drug container
112 front end
114 actuator element
116 needle shield
118 needle
120 pierceable portion
122 first end
124 second end
126 connecting means
128 needle cap
130 magazine
132 compartment
134 arrow
136 front end
138 rear end
140 spring
142 needle mount
144 moveable stopper
146 stopper mount
148 slit
150 reservoir
152 arrow
154 position
156 spring
158 spring
160 valve
162 spring
164 outer casing
166 inner casing

1-15. (canceled)

16. A medication delivery device, comprising
a housing;
a drug container holder arranged at least partly within the housing, wherein the drug container holder is configured to receive a plurality of single dose drug containers, each single dose drug container of the plurality of single dose drug containers configured to contain a liquid; and
an actuator element configured to separately administer a single dose of the liquid from each single dose drug container of the plurality of single dose drug containers.

17. The medication delivery device according to claim 16, wherein the actuator element is configured to separately administer the single dose through a hollow needle.

18. The medication delivery device according to claim 17, wherein the hollow needle comprises a connector configured to connect the hollow needle to each single dose drug container of the plurality of single dose drug containers.

19. The medication delivery device according to claim 18, wherein the connector comprises a bulge configured to engage a depression of each single dose drug container of the plurality of single dose drug containers to connect the hollow needle to each single dose drug container of the plurality of single dose drug containers.

20. The medication delivery device according to claim 17, wherein the hollow needle comprises a first end configured to penetrate a patient's skin and a second end configured to penetrate a pierceable portion of each single dose drug container of the plurality of single dose drug containers.

21. The medication delivery device according claim 20, wherein the actuator element is configured to move the hollow needle relative to each single dose drug container of the plurality of single dose drug containers such that the hollow needle penetrates the pierceable portion.

22. The medication delivery device according to claim 17, wherein the hollow needle is substantially U-shaped.

23. The medication delivery device according to claim 17, wherein the hollow needle comprises a longitudinal shape.

24. The medication delivery device according to claim 16, wherein the actuator element is configured to move a moveable stopper of each single dose drug container of the plurality of single dose drug containers to cause the liquid of the single dose drug container to be discharged from the single dose drug container.

25. The medication delivery device according to claim 24, wherein a movement of the moveable stopper reduces an inner volume of the single dose drug container.

26. A medication delivery assembly comprising the medication delivery device according to claim 16 and the plurality of single dose drug containers.

27. The medication delivery assembly according to claim 26, further comprising a magazine, wherein the plurality of single dose drug containers are arranged in the magazine.

28. The medication delivery assembly according to claim 26, further comprising a hollow needle.

29. The medication delivery assembly according to claim 28, wherein the hollow needle is configured to be removably connected to the housing.

30. The medication delivery assembly according to claim 26, wherein each single dose drug container of the single dose drug containers comprise a spring configured to bias the actuator element towards a start position in which administration of the single dose is prevented.

31. A method for administration a single dose of a liquid using the medication delivery device according to claim 16, comprising:
inputting one single dose drug container of the plurality of single dose drug containers into the drug container holder, wherein the one single dose drug container includes the liquid, and
operating the actuator element to administer the single dose of the liquid from the one single dose drug container.

32. A medication delivery assembly comprising:
a plurality of single dose drug containers; and
a medication delivery device comprising:
a housing;
a drug container holder arranged at least partly within the housing, wherein the drug container holder is configured to receive the plurality of single dose drug containers, each single dose drug container of the plurality of single dose drug containers configured to contain a liquid; and
an actuator element configured to separately administer a single dose of the liquid from each single dose drug container of the plurality of single dose drug containers through a hollow needle,
wherein the actuator element is configured to move the hollow needle relative to each single dose drug container of the plurality of single dose drug containers such that the hollow needle penetrates a pierceable portion of each single dose drug container of the plurality of single dose drug containers, and
wherein each drug container of the single dose drug containers comprise a biasing element configured to bias the actuator element towards a start position in which administration of the single dose is prevented.

33. The medication delivery assembly according to claim 32, wherein the actuator element is configured to move a moveable stopper of each single dose drug container of the plurality of single dose drug containers so as to cause the liquid of the single dose drug container to be discharged from the single dose drug container.

34. The medication delivery assembly according to claim 33, wherein the hollow needle is U-shaped.

35. A method for manufacturing a medication delivery device, comprising:
providing a housing,
arranging a drug container holder at least partly within the housing, wherein the drug container holder is configured to receive a plurality of single dose drug containers, each single dose drug container configured to contain a liquid, and
mounting an actuator element configured to separately administer a single dose of the liquid from each single dose drug container of the plurality of single dose drug containers.
